(19) United States
(12) Patent Application Publication
(10) Pub. No.: US 2007/0289697 A1
(43) Pub. Date: Dec. 20, 2007

(54) Method For Producing Blanks For Disposable Absorbent Articles

(75) Inventors: Hans Een, Molnlycke (SE); Kent Hermansson, Vastra Frolunda (SE)

Correspondence Address: BUCHANAN, INGERSOLL & ROONEY PC, POST OFFICE BOX 1404, ALEXANDRIA, VA, 22313-1404, US

(73) Assignee: SCA HYGIENE PRODUCTS AB, Goteborg (SE)

(21) Appl. No.: 11/845,139

(22) Filed: Aug. 27, 2007

Related U.S. Application Data
(62) Division of application No. 10/383,615, filed on Mar. 10, 2003.
(60) Provisional application No. 60/362,544, filed on Mar. 8, 2002.

(51) Int. Cl. A61F 13/15

(52) U.S. Cl. 156/164

(57) ABSTRACT

A process for manufacturing blanks for disposable pants includes feeding a web having a longitudinal direction and a transverse direction, cutting the web and elastics into a plurality of first and second pant blanks, wherein each of the first pant blanks has a front part extending along a first edge and a back part extending along a second edge and each of the second pant blanks has a front part extending along the second edge and a back part extending along the first edge, wherein the first and second pant blanks are arranged in alternating sequence along the longitudinal direction and wherein the back part of each of the pant blanks is longer than the front part of each of the pant blanks, and cutting first and second cut-out portions from the web, wherein each of the pant blanks is arranged between one of the first cut-out portions and one of the second cut-out portions.

### CROSS-REFERENCE TO RELATED APPLICATIONS

The present application is a divisional of U.S. Ser. No. 10/383,615, which claims the benefit of U.S. Provisional Application No. 60/362,544, filed in the United States on Mar. 8, 2002, the entire contents of which are hereby incorporated herein by reference.

### BACKGROUND OF THE INVENTION

1. Technical Field

The invention relates to a process for manufacturing blanks for disposable articles, such as a pant or pant diaper, and for manufacturing such articles.

2. Background Art

It has long been known to produce disposable pants and disposable pant diapers in an automated process plant. Production speed is important, because the more disposable pants that are produced per time unit, the shorter the time period required to obtain the wanted number of finished disposable pants. The quality of the produced pants is naturally another highly important issue due to the demands from the customers, and because the production quality reflects directly upon the production economy. By not having to throw away defective disposable pants, a manufacturer may save a great deal of money. Another important factor is the wish to minimize the waste, since the manufacturer normally has to pay a fee for waste management, i.e., for getting rid of the waste. Furthermore, the waste itself represents an expense in the form of loss of material.

In addition, there exists a demand from customers that the disposable pants or disposable pant diapers be as comfortable as possible. In order to increase the comfort the customers want, disposable pants or disposable pant diapers, during use, do not have seams running over the hips of the user, on the waist sides or in a seam in the crotch region.

In order to avoid seams which during use will be positioned over the hips of the user, it is previously known to produce disposable pants having waist side seams displaced towards the front of the disposable pants. The disposable pants are cut from a web and a number of cut-out portions are cut out from the web to form leg openings in the pants. The web is also cut in such way as to form a back body portion of the pant and a front body portion. The back body portion and the front body portion are bonded to each other, forming a waist-encircling part. In order to get the waist side seams displaced towards the front body portion, some of the width of the front body portion is cut away before the back body portion is attached to the front body portion. Even though such production gives a disposable pant with waist side seams displaced towards the front body portion, the cutting of the front body portion results in a considerable amount of waste as well as a time-consuming process step.

Alternative process methods have been suggested in order to overcome the problems with undesirably placed seams and unreasonable amounts of waste. The most common way to reduce waste is to manufacture disposable pant in parts and to assemble the parts in a marriage point further along the production line. One problem with such methods is that the production speed is decreased and the risk for assembling the parts wrong is evident. Furthermore, assembling different parts gives rise to a number of not wanted seams, for example in the crotch area of a user.

In order to overcome the problem with assembling parts, a number of solutions have been proposed. GB 2257347, for example, shows a process for manufacturing a disposable pant. A web is continuously fed in its longitudinal direction, and is divided in an upper and a lower part along a longitudinal centre line, which parts are bonded to each other along the longitudinal centre line. The upper part is intended to form a back body portion of the pant and the lower part is intended to form a front body portion of the pant. The back body portion consists of an essentially non-stretchable material, and the front body portion consists of a pre-stretched material. In the process, a number of cut-out portions are cut out of the web to form each of the leg openings of the pant. The cut-out portions have a desired size and shape and are symmetric with respect to a line running in the transverse direction of the web, centrally through the upper and lower part of each pant blank. The web is then folded in two along the longitudinal centre line. The folded web is then bonded and cut to form the individual pants. The pre-stretched front body portion contracts in such a way that the back body portion stretches over the waist sides of the user when the pant is in use, offering the user a more comfortable pant with no seams positioned directly on the waist sides.

Even though the pant described in GB 2257347 is produced with minimum waste and has no seams on the waist sides, there still exist some problems related both to the production process and to the pant itself. In order to get the seams away from the waist sides, the front body has to be made of a pre-stretched material, and the back body of a different material. Accordingly, the production line has to include a first slow step with a pre-stretching of the material in the front body portion and a second step of bonding that material to another material in the back body portion with a seam that would land in the crotch region of a user during use. If these two steps could be eliminated, the production process would be much faster and more reliable, and the seam from the bonding in the second step would not appear in the crotch area of the user when the pant is in use. The elimination of such a seam in the crotch area would of course make the pant more comfortable for a user.

Thus, there still exists a need for an improved manufacturing process for disposable pants. The improvements should aim at giving the manufacturer a better and cheaper product, specifically by offering an efficient production line with a high production volume and with as little waste as possible, and also by resulting in a disposable pant being more comfortable for the user.

### SUMMARY

An embodiment according to the invention will have waist side seams displaced towards the front portion of the pant, and no seam in the crotch region of the pant.

A typical disposable pant, or pant diaper, comprises a liquid-impervious back sheet facing away from a user during use, and a liquid-pervious top sheet facing the user during use, and one or more absorbent bodies arranged therebetween. However, the disposable pant may also be intended for use with one or more absorbent inserts.

The back sheet may be of any liquid-impermeable material or material that has been made liquid-impermeable, e.g., a treated nonwoven, or any plastic material, or a laminate of nonwoven.

The top sheet may be of any inherently liquid-permeable material or of a material that has been treated to be liquid-permeable, e.g., nonwoven, textiles or perforated plastic film.

The absorbent bodies may be of any liquid retaining material, or layers of liquid retaining material, or a combination of liquid distributing material and liquid retaining material. The absorbent body may also comprise a superabsorbent which is an absorbent polymer material capable of absorbing fluids in an amount corresponding to several times the dry weight of the superabsorbent and which upon absorbent swells into a fluid-containing gel.

One embodiment of the invention relates to a process for manufacturing a disposable pant originating from a pant blank, which disposable pant comprises a front portion, a back portion and a central portion extending between the front portion and the back portion. The disposable pant also comprises side edges forming leg openings of the pant and thus delimiting the disposable pant in that area that is intended to surround the legs of a user.

The pant blank comprises a front part and a back part and a central part extending therebetween, which parts correspond to the front portion, the back portion and the central portion of the final disposable pant. The process for manufacturing the disposable pant according to one aspect of the invention comprises continuously feeding a web along a path, said web having a longitudinal direction coinciding with said path and a transverse direction perpendicular to said longitudinal direction, said web having a first edge and a second edge extending in said longitudinal direction and elastics extending continuously in the longitudinal direction, cutting the web and elastics into a plurality of first and second pant blanks, wherein each of the first pant blanks has a front part extending along the first edge and a back part extending along the second edge and each of the second pant blanks has a front part extending along the second edge and a back part extending along the first edge, wherein the first and second pant blanks are arranged in alternating sequence along the longitudinal direction and wherein the back part of each of the pant blanks is longer than the front part of each of the pant blanks, and cutting first and second cut-out portions from the web, wherein each of the pant blanks is arranged between one of the first cut-out portions and one of the second cut-out portions.

The pant blanks constitute the starting material when forming the disposable pants. The web may comprise one single layer of one material or a number of layers of the same or different materials, and dependent on the web features the pant blanks form different kinds of disposable pant. For example, absorbent bodies may be placed on the web which gives the possibility to manufacture disposable absorbent pants or pant diapers.

According to another aspect of the invention, a process for manufacturing a plurality of disposable pants, comprising forming a plurality of pant blanks according to the above-mentioned process, and wherein the pant blanks each are folded and the front parts of the first and second blanks are joined to the respective back parts of the first and second blanks in such way that the back part of each first blank is reclosably or permanently attached to the front part of each first blank and the back part of each second blank is reclosably or permanently attached to the front part of each second blank, thereby forming the disposable pants.

The pant blanks along the web of the preferred embodiment may be described as having a first distance defining the length of a first back part on every first blank along the first end edge, and a second distance defining the length of a first front part on every first blank along the second end edge, and a third distance defining the length of a second front part on every second blank along the first end edge, and a fourth distance defining the length of a second back part on every second blank along the second end edge.

Preferably, the process for manufacturing a disposable pant according to an embodiment of the invention includes cutting the web in such a way that the value of the first distance divided by the second distance and the value of the fourth distance divided by the third distance is in the range of 1.8-5.7.

Upon forming the disposable pant a seam may appear in the area where the back portion is joined to the front portion. In previously known transversal production methods for disposable pants, the length of the back part and the length of the front part along the production line is the same, i.e., the pant blanks lie next to each other, resulting in a seam on the disposable pant that is situated over the hip, at the side of the user. It is a desired feature to have a disposable pant with the seam displaced towards the front of the user, i.e., the seam is situated in such way that when the disposable pant is used, the seam is situated somewhere on the abdomen of the user.

According to an embodiment of the invention, due to the difference in the lengths according to the above, the back portion of the disposable pant will be wider than the front portion and the seam between the back portion and the front portion will be arranged towards the front of the disposable pant when in use.

According to one embodiment of the invention, a number of first cut-out portions of the web are cut out from the web along first portion cutting lines and a number of second cut-out portions of the web are cut out from the web along second portion cutting lines. The first and second cut-out portions are arranged at predetermined distances from each other in the longitudinal direction of the web and are preferably cut in such way that they form the side edges of the final product, i.e., the disposable pant.

The cut-out portions may be identical or different from each other, depending on the desired features of the disposable pant. The portion cutting lines respectively need not be one single cutting line, but may be a number of cutting lines that are connected. Such connected cutting lines may be straight or curved and at least one such cutting line may extend over an imaginary longitudinal center line, for example in a straight line crossing the longitudinal center line and being perpendicular to the longitudinal centre line or at another suitable angle.

Furthermore, the web is cut along first cutting lines, which first cutting lines each extend from the first end edge to a first point on the first portion cutting line. The web is also cut along second cutting lines, which second cutting lines each extend from the first end edge to a second point on the second portion cutting line. The web is also cut along third cutting lines, which third cutting lines each extend from the second end edge to a third point on the first portion cutting line. The web is also cut along fourth cutting lines, which fourth cutting lines each extend from the second end edge to a fourth point on the second portion cutting line.

The web does not have to be cut along the cutting lines in any specific order, but the web may be cut along any one or all of the first, second, third and fourth cutting lines simultaneously with a part of or the entire first and second portion cutting lines. Hence, one or all of the first, second, third and fourth cutting lines may coincide with at least a part of the portion cutting lines.

The first, second, third and fourth cutting lines may be straight or curved, or may comprise both straight and curved segments. In addition, the cutting lines may be perpendicular to the end edges of the web or at a different angle to the end edges of the web.

After the pant blanks are cut from the web, each pant blank is folded and the front part is joined to the corresponding second back part in such way that the back part of each of the pant blanks is reclosably or permanently attached to the front part in an overlapping manner at overlapping parts. The folded pant blanks thereby form the disposable pants.

The expression “overlapping manner” refers to when an outside of one sheet is placed against the inside of another sheet, or vice versa. Hence, according to one embodiment of the invention, the inside of the back portion is placed over the outside of the front portion, or vice versa.

The overlapping parts are defined by predetermined distances from the first cutting line to the third cutting line and the second cutting line to the fourth cutting line in the overlapping parts, i.e., the overlapping parts will be those parts of the pant blank that constitute the distance between the first cutting line and the third cutting line when the back part is folded over the front part.

For the first pant blank, the first cutting line is lying over the first front part and the second cutting line is lying over the first front part. For the second pant blank, the third cutting line is lying over the second front part and the fourth cutting line is lying over the first front part.

However, it is possible to manufacture a disposable pant in accordance with the invention, where the front part is overlapping the back part. The front part may be either reclosably or permanently attached to the back part in an overlapping manner.

If the disposable pant is supposed to be reclosable, the overlapping parts may be equipped with hook and loop materials or any other suitable reclosable means. If the overlapping parts are to be attached permanently, a seam may be applied in order to secure the overlapping parts against each other. The seam may be the result of a sealing operation including welding, hotmelting, ultrasound, gluing, or any other suitable method. There may also be a combination of an openable seam, i.e., a seam that is possible to tear up or break and a reclosable part with reclosable fastening means. The seam that is openable may help to keep the pant together when a user is putting the pant on. Such a seam may be placed on the inside of the reclosable fastening means.

The folding of the pant blank is preferably done over a roller that includes a folding mechanism, for example gripping means in the very corners of the pant blank and a dolly or holding tool that the pant blank can be folded over.

One feature of the embodiment of the invention is that, due to the first distance being greater than the second distance and the fourth distance being greater than the third distance, the overlapping parts will be arranged towards the front portion, i.e., any seams or reclosable parts will be arranged towards the front portion of the disposable pant. The arrangement of the overlapping parts towards the front part increases the comfort for the user, since the front part is less exposed to pressure from outer clothing, and thereby less likely to cause the user discomfort. Additionally, it is easier to handle a reclosable diaper when the overlapping parts are placed at the front portion, because children tend to be handled lying on their backs, and grown ups using a disposable pant can more easy handle overlapping parts on the front part than on the sides.

In accordance with one embodiment of the invention, the cut-out portions and the pant blanks are advantageously cut in such way that every first and second pant blanks achieve similar or identical shapes.

The first and second cut-out portions may, for example, each have a shape describing two opposing half circles with two parallel lines connecting the half circles. However, the cut-out portions may each have an oval shape, rectangular shape, square shape, or any other suitable shape for a leg opening thereby giving the desired feature of the disposable pant with different shapes of the side edges of the disposable pant.

When manufacturing a disposable absorbent pant or an absorbent article in general using the process according to an embodiment of the invention, absorbent bodies are placed along the web with a fifth predetermined distance between the absorbent bodies. The disposable absorbent pant may of course be equipped with a number of different absorbent layers and a number of other layers such as liquid distributing layers as well as layers especially suitable to be placed against the skin of a user, for example a liquid-permeable and skin friendly material such as nonwoven.

The absorbent bodies do not have to be placed on the web before the cutting but may be placed in or on each of the pant blanks at any time during the manufacturing process. For example, the absorbent bodies may be placed in the pant blanks just before the folding of the pant blanks.

In another embodiment of the invention, the absorbent bodies are in the form of a unit that can be applied on that side of the disposable pant that faces the user during use. Such an absorbent unit may comprise a liquid-impervious layer, a liquid-pervious layer and one or more absorbent layers therebetween. The absorbent unit may also be equipped with fasteners on that side of the liquid-impervious layer that lies adjacent to the disposable pant, in order to keep the absorbent unit in place in the disposable pant. The disposable unit may also or only comprise fasteners on that part of the liquid-permeable layer that lies adjacent the skin of a user during use, in order to keep the absorbent unit in place against the body of the user. The latter fasteners may comprise different kinds of skin care products such as ointment, lotion, or the like. The absorbent unit may also be equipped with any type of conventional liquid retaining devices, such as standing gathers, side barriers, leg elastics, humps, and superabsorbent materials.

Depending on the features of the absorbent unit, the disposable pant, i.e., the web, may be of any suitable material. For example, if the absorbent unit has a liquid-impervious layer and a liquid retaining system, the web making up the pant may be of a suitable breathable material, and if the absorbent unit lacks a liquid-impervious layer, the web may be of any suitable liquid-impermeable material.

However, the absorbent units may also be placed on the web or between layers of the web during the manufacturing process.

Another way to manufacture a disposable pant having absorbent features, or other features, is to form the web in such way that it comprises all, or some, of the materials that correspond to the desired features of the final product, already when the web is fed into the process. For example, the web may comprise a liquid impervious layer, a liquid pervious layer and an absorbent body therebetween.

In order to get a desired shape of the disposable pant, elastics may be applied on the web. It is previously known to use elastics when forming a disposable pant, for example when forming leg elastics, liquid barriers, humps and waist elastics. One advantage with the present invention is that the forming of the pant blanks correspond to the shape of the final disposable pants, which offers unique possibilities to apply elastics to the web in any desired pattern. In previously known processes for manufacturing disposable pants, the disposable pant is assembled from at least two parts that are bonded together in the central portion. Such a construction limits the way in which elastic elements can be arranged along the leg openings or the waist opening of the pant or pant diaper.

The elastic may thus be applied or arranged in a number of different patterns, e.g., in an anti-phase zigzag pattern, or in an anti-phase sinusoidal form. Anti-phase means that two elastics start from starting points on opposing sides of a center line running in the longitudinal direction of the product web and run in opposing patterns along the web. The elastics may be applied to the web in such a way that the different elastics cross each other in a small loop in the central portion, or does not cross at all, or in such a way that the elastics run from one side of the web to the other. Elastics may also be applied to the back part forming waist elastics and/or to the front part forming waist elastics and/or on the parts that form the side portions or leg openings of the final product.

According to another aspect of the invention, a process for manufacturing a plurality of blanks for disposable pants comprises continuously feeding a web along a path, said web having a longitudinal direction coinciding with said path and a transverse direction perpendicular to said longitudinal direction, said web having a first edge and a second end edge extending in said longitudinal direction, and cutting the web into a plurality of first and second pant blanks, wherein each of the first pant blanks has a front part extending along the first edge and a back part extending along the second edge and each of the second pant blanks has a front part extending along the second edge and a back part extending along the first edge, wherein the first and second pant blanks are arranged in alternating sequence along the longitudinal direction and wherein the back part of each of the pant blanks is longer than the front part of each of the pant blanks.

According to another aspect of the invention, a process for manufacturing blanks for disposable pants comprises continuously feeding a web along a path, said web having a longitudinal direction coinciding with said path and a transverse direction perpendicular to said longitudinal direction, said web having a first edge and a second end edge extending in said longitudinal direction, cutting a plurality of first and second cut-out portions from the web in alternating sequence along the longitudinal direction of the web, forming a back part of each of a plurality of first pant blanks by cutting a first line between the first edge and one of the first cut-out portions and cutting a second line between the first edge and one of the second cutout portions, forming a front part of each of the first pant blanks by cutting a third line between the second edge and one of the first cut-out portions and by cutting a fourth line between the second edge and one of the second cutout portions, wherein a plurality of second pant blanks are formed in alternating sequence with the plurality of first pant blanks along the longitudinal direction of the web such that the front part of each of the second pant blanks is formed by the first and second cutting lines and the back part of each of the second pant blanks is formed by the third and fourth cutting lines, and wherein the back part of each of the pant blanks is longer than the front part of each of the pant blanks.

According to another aspect of the invention, a process for manufacturing a plurality of disposable pants comprises forming a plurality of pant blanks according to the above-mentioned process, folding the plurality of pant blanks along a center line extending in the longitudinal direction such that each of the front parts are attached to a respective back part, and reclosably or permanently attaching the front parts to the respective back parts.

The invention also includes a product manufactured by the process according to the above.

### BRIEF DESCRIPTION OF DRAWINGS

The invention will below be explained in some embodiments with the aid of figures.

FIG. 1 shows a web on a production line according to an embodiment of the invention.

FIG. 2 shows an outlined flowchart of the process.

FIG. 3 shows a disposable pant according to an embodiment of the invention.

### DETAILED DESCRIPTION OF THE PREFERRED EMBODIMENTS

A production process for manufacturing embodiments of a disposable pant according to the invention will be shown, which embodiments shall not be seen as limiting the invention, but merely as examples of different ways to use the process.

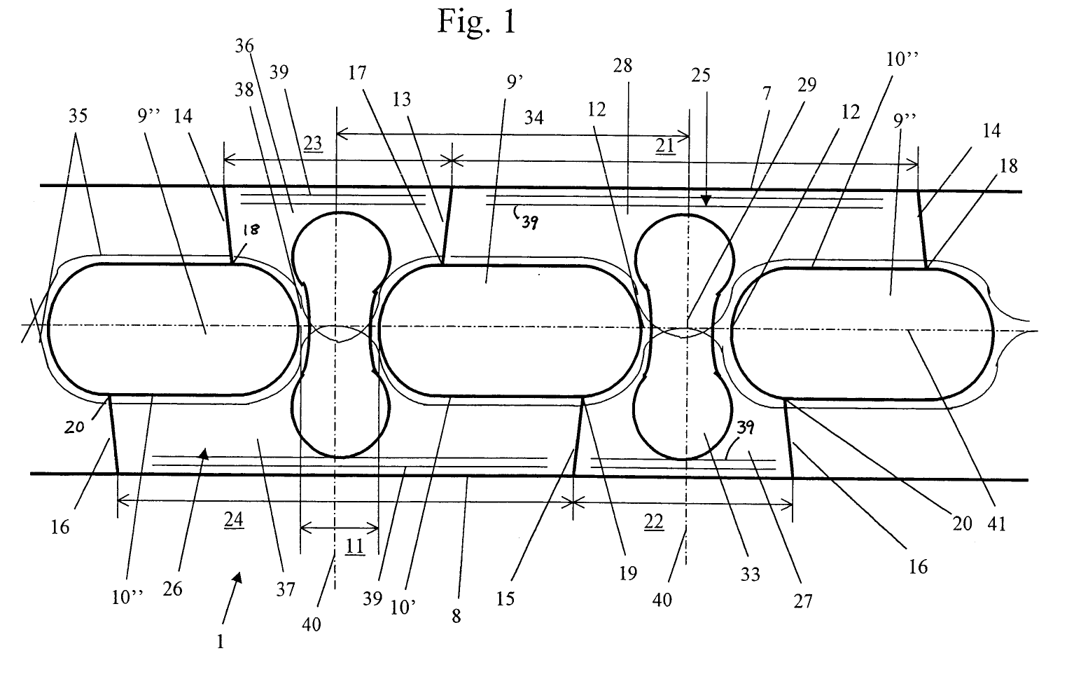

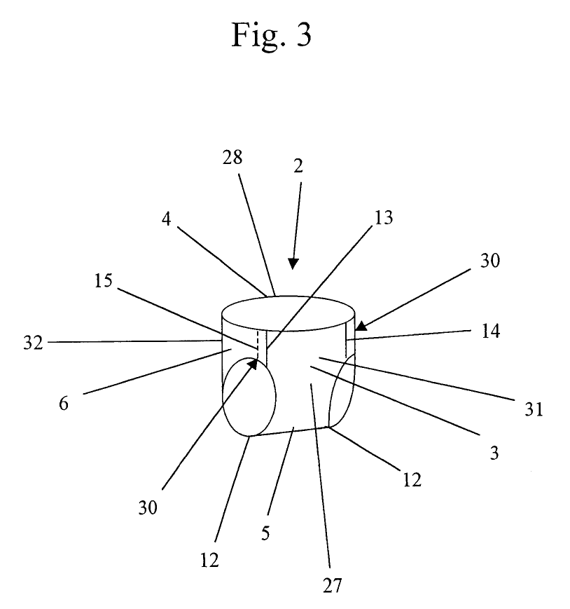

FIG. 1 shows a web 1 on a production line according to an embodiment of the invention, in a process for manufacturing a disposable pant 2 (see FIG. 3), for example, pant diapers.

The process is intended for use in manufacturing an embodiment of a disposable pant 2 comprising a front portion 3 and a back portion 4 and a central portion 5 extending therebetween, which central portion 3 extends in the crotch area of a user during use. The disposable pant 2 also comprises two side portions 6 extending between the front portion 3 and the back portion 4 over the waist sides of a user when in use.

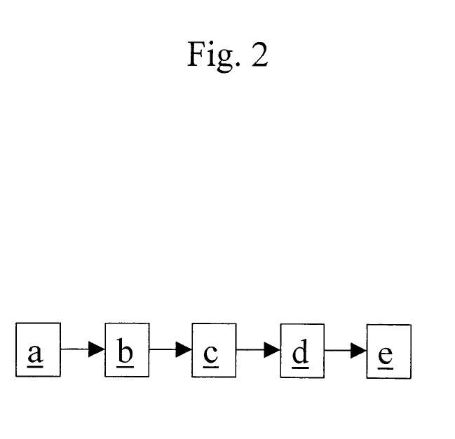

FIG. 2 shows one embodiment of the invention, where the process for manufacturing the disposable pant 2 comprises the steps of (see FIG. 2):

a) Continuously feeding a web 1 delimited by two parallel longitudinally extending first and second end edges 7, 8 in a longitudinal direction thereof. The longitudinal direction of the web coincides with the feed direction, or travel direction of the web. The web may be of any material or mix of materials suitable for the purpose, e.g., a liquid-impervious material suitable for making up the outside of a disposable pant or disposable pant diapers. When using the process for manufacturing disposable pant diapers the web may comprise a liquid impervious layer and a liquid pervious layer and one or several absorption layers arranged therebetween. Also, it is possible to use a liquid-pervious material for the web 1, and to place on the web an absorbent unit formed by a liquid-impervious layer and a liquid-pervious layer, and an absorbent body arranged therebetween.

b) Cutting a number of first and second cut-out portions 9′, 9″ from the web 1 along first and second portion cutting lines 10′, 10″, which cut-out portions 9′, 9″ are arranged at a predetermined portion distance 11 from each other. The portion distance 11 is preferably the distance between side edges 12 of the central portion 5 of the disposable pant 2, which side edges 12 delimits the width of the central portion 5 of the disposable pant 2 in its unfolded position. The first and second portion cutting lines 10′, 10″ each describe the part of the disposable pant that is intended to be leg openings.

c) Cutting the web 1 along first cutting lines 13, which first cutting lines 13 each extend from the first end edge 7 to a first point 17 on the first portion cutting line 10′, and the web 1 is also cut along second cutting lines 14, which second cutting lines 14 each extend from the first end edge 7 to a second point 18 on the second portion cutting line 10″, the web 1 is also cut along third cutting lines 15, which third cutting lines 15 each extend from the second end edge 8 to a third point 19 on the first portion cutting line 10′, and also cut along fourth cutting lines 16, which fourth cutting lines 16 each extend from the second end edge 8 to a fourth point 20 on the second portion cutting line 10″. In FIG. 1, the first, second, third, and fourth cutting lines 13, 14, 15, 16 are shown as being at an angle to the end edges 7, 8 of the web 1, but the cutting lines 13, 14, 15, 16 may be perpendicular to the end edges 7, 8.

The first and second cutting lines 13, 14 recur in a pattern along the longitudinally extending first end edge 7 with a first cutting line 13 arranged at a predetermined first distance 21 to a second cutting line 14, and the first cutting line 13 arranged at a predetermined third distance 23 to another second cutting line 14, with the first distance 21 being greater than the third distance 23.

The third and fourth cutting lines 15, 16 recur in a pattern along the longitudinally extending second end edge 8 with a third cutting line 15 arranged at a predetermined second distance 22 to a fourth cutting line 16, and the third cutting line 15 arranged at a predetermined fourth distance 24 to another fourth cutting line 16, with the fourth distance 24 being greater than the second distance 22.

Furthermore, the first distance 21 is greater than the second distance 22, and the fourth distance 24 is greater than the third distance 23.

The first, second, third, and fourth cutting lines 13, 14, 15, 16 and the cut-out portions 9′, 9″ divide the web into a number of first and second pant blanks 25, 26. Here, pant blanks 25, 26 are the disposable pants 2 before forming any side panels, and which disposable pant is in an unfolded fully planar state. The first pant blank 25 has a first front part 27, a first back part 28 and a first central part 29, which parts 27, 28, 29 correspond to the front portion 3, back 4 portion and the central portion 5 of the finished disposable pant 2. The second pant blank 26 has a second front part 36, a second back part 37 and a second central part 38, which parts 36, 37, 38 correspond to the front portion 3, back 4 portion and the central portion 5 of the final disposable pant 2.

d) After the cutting according to steps b and c, folding each of the pant blanks 25, 26 with their back parts 28, 37 folded over the front parts 27, 36 in predetermined zones, overlapping parts 30. The first pant blank 25 is folded such that the overlapping parts 30 are defined by predetermined distances from the first cutting line 13 to the third cutting line 15 and the second cutting line 14 to the fourth cutting line 16 in the overlapping parts, i.e., the overlapping parts 30 will be those parts of the first pant blank 25 that constitute the distance between the first cutting line 13 and the third cutting line 15 when the first back part 28 is folded over the first front part 27 such that the first cutting line 13 is lying over the front part, and vice versa, for the second cutting line. A portion of the first back part 28 forms the side portions 6 of the final disposable pant, i.e., the side portions 6 mainly consist of parts of the first back part 28, but may also include parts of the first front part 27, depending on the first and second distances 21, 22. The second pant blank 26 is folded in a corresponding way as the first pant blank 25.

e) Joining folded pant blank 25, 26 in the overlapping parts, either by a reclosable material or by a permanent seam.

The above described process for manufacturing of a disposable pant 2, can more easily be described as continuously cutting a number of first and second pant blanks 25, 26 from a web 1 in such way that every second pant blank lies parallel with but with an opposite orientation to every first pant blank along the longitudinal direction of the web 1, i.e. a first pant blank 25 lies parallel to and turned in an opposite direction to a neighbouring second pant blank. The first distance 21 marks the length in the travelling direction of the web 1, of a first back part 28 on every first pant blank, and the fourth distance 24 marks the length of a second back part 37 on every second pant blank. The second distance 22 marks the length of a first front part 27 on every first pant blank, and the third distance 23 marks the length of a second front part 36 on every second opposing pant blank.

When the first pant blank 25 is folded and bonded according to steps d and e above, the first back part 28 is bonded to the first front part 27 in the overlapping parts 30. The first back part 28 bonded to the first front part 27 form the side portions 6 in the disposable pant. The side portions 6 are defined by that area of the disposable pant that during use extends over the waist sides of a user, i.e., between a front panel 31 in the front portion 3 and a back panel 32 in the back portion 4 in the disposable pant 2. The second pant blank 26 is preferably folded and bonded in a corresponding way as the first pant blank 25, but may be folded and bonded in another way. For example, the first pant blank 25 may be folded and bonded with reclosable means while the second pant blank may be folded and bonded with a permanent seam.

If the disposable pant is supposed to be reclosable, the overlapping parts may be equipped with hook and loop materials or any other suitable reclosable means. If the overlapping parts are to be attached permanently, a seam may be applied in order to secure the overlapping parts against each other. The seam may be the result of an operation such as welding, hotmelting, ultrasound, gluing, stitching, or any other suitable method. There may also be a combination of a seam that is possible to tear up and a reclosable part with reclosable fastening means. The seam that is possible to tear up may help to keep the pant together when a user is putting the pant on. Such a seam may be placed on the inside of the reclosable fastening means.

Since the first distance 21 exceeds the second distance 22, the bonding area in the overlapping part 30 in the side portions 6 will be arranged towards the front panel 31, which is a great advantage compared to having side portions with bonding areas at the waist sides of a user, which is common in the prior art.

The forming of a second pant blank 26 into a disposable pant can, of course, be made in complete analogy with the above description in connection with the first pant blank.

The disposable pant according to an embodiment of the invention has been described as having overlapping bonding areas, but it is also possible to bond the back part to the front part with non-overlapping seams, i.e., the inside of the back part is bonded to the inside of the front part.

Thus, one great benefit of one aspect of the invention is that the disposable pant can be made from one single web without using, e.g., elastics in the front portion in order to draw the overlapping parts 30, i.e., the bonding areas, towards the front portion. The use of one single web, i.e., one pant blank forming a disposable pant, also eliminates a bonding area in the central part of the disposable pant. Such centrally located bonding area is common in prior art and is known to cause irritation and discomfort for a user in the crotch area.

The first and third cutting lines 13, 15 are preferably mirror images of the second and fourth cutting lines respectively over a transverse imaginary center line 40, which is shown in FIG. 1. The cut-out portions 9 are preferably symmetrically aligned with a longitudinal center line 41, see FIG. 1, but may of course be cut from the web in another pattern dependent on the desired features of the final product. The symmetrically cut pant blanks described above advantageously yields a number of first and second pant blanks that have the same shape, but the cutting line and the cutting portions may be cut in such a way that the first and second pant blanks have different shapes.

In one embodiment of the invention, the process is used for manufacturing a disposable pant diaper, see FIG. 1, where absorbent bodies 33 are placed along the web with a predetermined fifth distance 34 between centers of the absorbent bodies. The absorbent bodies may be placed before the cutting, during the cutting or after the cutting of the web, dependent on the process. One example may be that the absorbent bodies 33 are placed on a layer consisting of a liquid impermeable material, after which a liquid permeable layer is placed over the absorbent bodies, which layers and absorbent bodies form the web.

The absorbent bodies each may be in any suitable shape, for example a rectangular shape or, as is shown in FIG. 1, an hourglass shape.

In one embodiment of the invention, see FIG. 1, elastics 35, 39 are applied to the web on the back parts, front parts and on either side of each of the cut-out portions 9′, 9″ partly along the end edges 7, 8 and also in the central parts 29, 38 along the side edges 12. The elastics 35 that are placed on either side of the cut-out portions 9′, 9″, and along the side edges 12 form leg elastics around the legs of a user during use. Preferably, the elastics 35 cross each other in a small loop in the central portion 29, 38 on every pant blank 25, 26. The elastics may be made inactive by cutting the elastics, a so called snap back procedure.

In another embodiment (not shown), the elastics are applied as described as above with the exception that the elastics do not cross in the central portion but two loops lie close to each other in the central portion of every pant blank.

The elastics may also be applied to the web as described as above but in a pattern where the elastics cross each other somewhere along the side edges and where the two loops are placed in the front part and the back part respectively on every pant blank such that there will be no elastics in the central portions.

Yet another possibility to apply elastics to the pant blanks is to place a first elastic means in a longitudinal sine curve that follows one side of the cut out portion and the side edge on one pant blank and that follows the other side of the cut out portion on the adjacent pant blank, and another elastic in a longitudinal sine curve following the opposite sides of said portions and the corresponding side edges.

The elastics may form leg elastics such as raised barriers also known as standing gathers, or side barriers, or other suitable liquid retaining means. Thus, the elastics are advantageously placed along the edges of the cut out portions (e.g., along the portion cutting lines 10′, 10″ or part of the portion cutting lines 10′, 10″ e.g., the side edges 12) that form the part of the pant diapers that is intended to surround at least parts of the legs of a user. It is also possible to add separate leg elastics in the central portions 5, for example, in the form of cuffs. Additionally, elastics may be applied to the pant blanks such that the elastics follow the portion cut out lines that lie essentially parallel to the end edges and which elastics cross the front parts and back parts respectively on the pant blanks.

In further embodiments, the elastics 35 may be placed in a number of different patterns, e.g., anti-phase zigzag pattern or anti-phase sinusoidal form. Anti-phase means that two elastics start from opposing starting points. The elastics may be applied to the web in such a way that the two different elastics cross each other in the central portion where the elastics run from one side 7, 8 of the web 1 to the other between the cut-out portions 9′, 9″, i.e., in the central parts 29, 38.

The elastics may also be applied in different forms on the back part forming waist elastics and/or on the front part forming waist elastics and/or on the parts that form the side portions of the final product. It is also possible to combine the application of separate leg elastics with separate waist elastics 39 (FIG. 1). The waist elastics are preferably of a such kind that the elastics contract the pant blank in the feeding direction of the web, and the leg elastics contract the finished product in a direction around the legs of a user, i.e., along the side edges. It is also possible to combine all of the above mentioned methods for applying elastics. The elastics may advantageously have different features on different places in the disposable pant, e.g., different tension and/or may be of different elastic materials or of different composition.

The elastics 35 intended for leg elastics are preferably of a thread type laid out in a curved pattern according to any conventional method and may be of an all-elastic material, but may also comprise a sheet, for example a nonwoven with elastic threads or strips with suitable elasticity, applied in suitable places along the sheet. Such strips or sheets are preferably used when placing waist elastics 39 preferably along the feeding direction of the web. The sheet may be a fabric or a nonwoven or any other suitable material. The elastics may also be applied on a separate web sheet, which web sheet will be applied on the web 1. If a liquid permeable top sheet has been applied to the pant blanks over the absorbent bodies, it is also possible to apply elastics forming side barriers on the top sheet.

Since the process is supposed to be a continuous process, it is advantageous to let the web run via different rollers equipped for different purposes. The cutting may, for example, be made by running the web through a pair of rollers with cutting tools. The folding of the pant blanks may be done by using a roller equipped with suction devices that are intended to hold the very corners of the pant blanks when the pant blanks are folded by, for example, mechanical arms. The joining of the back part to the front part may be done by letting the web pass through a pair of rollers equipped with, for instance, welding tools or gluing tools, and a dolly that separates the back part from the front part leaving only the bonding area to be bonded.

The invention shall not be seen as limited by the above-described embodiments, but all the embodiments may be combined within the scope of the claims.

What is claimed is:

1. A process for manufacturing a plurality of blanks for disposable pants, said process comprising:
continuously feeding a web along a path, said web having a longitudinal direction coinciding with said path and a transverse direction perpendicular to said longitudinal direction, said web having a first edge and a second edge extending in said longitudinal direction and elastics extending continuously in the longitudinal direction,
cutting the web and elastics into a plurality of first and second pant blanks, wherein each of the first pant blanks has a front part extending along the first edge and a back part extending along the second edge and each of the second pant blanks has a front part extending along the second edge and a back part extending along the first edge, wherein the first and second pant blanks are arranged in alternating sequence along the longitudinal direction and wherein the back part of each of the pant blanks is longer than the front part of each of the pant blanks, and
cutting first and second cut-out portions from the web, wherein each of the pant blanks is arranged between one of the first cut-out portions and one of the second cut-out portions.

2. The process according to claim 1, wherein the first cut-out portions are substantially similar to the second cut-out portions.

3. The process according to claim 1, wherein the back part of each of the first pant blanks is defined at one end by a first cutting line between the first edge and one of the first cut-out portions and at a second end by a second cutting line between the first edge and one of the second cut-out portions.

4. The process according to claim 3, wherein the front part of each of the first pant blanks is defined at one end by a third cutting line between the second edge and one of the first cut-out portions and at a second end by a fourth cutting line between the second edge and one of the second cut-out portions.

5. The process according to claim 4, wherein the front part of each of the second pant blanks is defined at one end by the first cutting line between the first edge and one of the first cut-out portions and at a second end by the second cutting line between the first edge and one of the second cut-out portions.

6. The process according to claim 5, wherein the back part of each of the second pant blanks is defined at one end by the third cutting line between the second edge and one of the first cut-out portions and at a second end by the fourth cutting line between the second edge and one of the second cut-out portions.

7. The process according to claim 6, further comprising:
folding the plurality of pant blanks along a center line extending in the longitudinal direction such that each of the front parts are attached to a respective back part, and
reclosably or permanently attaching the front parts to the respective back parts.

8. A process for manufacturing blanks for disposable pants, said process comprising:
continuously feeding a web along a path, said web having a longitudinal direction coinciding with said path and a transverse direction perpendicular to said longitudinal direction, said web having a first edge and a second edge extending in said longitudinal direction, and elastics extending continuously in the longitudinal direction, the elastics including looped portions,
cutting a plurality of first and second cut-out portions from the web in between the looped portions of the elastics in alternating sequence along the longitudinal direction of the web,
forming a back part of each of a plurality of first pant blanks by cutting a first line between the first edge and one of the first cut-out portions and cutting a second line between the first edge and one of the second cut-out portions,
forming a front part of each of the first pant blanks by cutting a third line between the second edge and one of the first cut-out portions and by cutting a fourth line between the second edge and one of the second cut-out portions,
wherein a plurality of second pant blanks are formed in alternating sequence with the plurality of first pant blanks along the longitudinal direction of the web such that the front part of each of the second pant blanks is formed by the first and second cutting lines and the back part of each of the second pant blanks is formed by the third and fourth cutting lines, and
wherein the back part of each of the pant blanks is longer than the front part of each of the pant blanks.

9. The process according to claim 8, wherein the first cut-out portions are substantially similar to the second cut-out portions.

10. The process according to claim 8, further comprising:
folding the plurality of pant blanks along a center line extending in the longitudinal direction such that each of the front parts are attached to a respective back part, and
reclosably or permanently attaching the front parts to the respective back parts.

11. A process for manufacturing disposable pants, said process comprising:
continuously feeding a web along a path, said web having a longitudinal direction coinciding with said path and a transverse direction perpendicular to said longitudinal direction, said web having first and second edges extending in said longitudinal direction and elastics extending continuously on the web substantially in the longitudinal direction,
cutting the web into a plurality of first and second pant blanks, each of the first and second blanks having a front part and a back part, said front and back parts being arranged in sequence in said transverse direction of said web, wherein the cutting is performed in such a way that the blanks are aligned in said longitudinal direction of said web with every second blank being rotated 180° with respect to every first blank, and cutting the web includes cutting the elastics;
folding the front and back parts of each blank in an overlapping manner; and
reclosably or permanently attaching the front parts to the respective back parts.

12. The process according to claim 11, wherein:
the web is cut along first cutting lines, which first cutting lines each extend from the first end edge to a first point on the first portion cutting line,
the web is cut along second cutting lines, which second cutting lines each extend from the first end edge to a second point on the second portion cutting line,
the web is cut along third cutting lines, which third cutting lines each extend from the second end edge to a third point on the first portion cutting line, and
the web is cut along fourth cutting lines, which fourth cutting lines each extend from the second end edge to a fourth point on the second portion cutting line.

13. The process according to claim 12, wherein one or more of the first, second, third and fourth cutting lines coincide with at least a part of one or both of the first and second portion cutting lines.

14. The process according to claim 11, wherein absorbent bodies are placed in each of the pant blanks.

15. The process according to claim 11, wherein the web includes a liquid impervious layer, a liquid pervious layer, and an absorbent body therebetween.
